(19) United States
(12) Patent Application Publication
(10) Pub. No.: US 2008/0228235 A1
(43) Pub. Date: Sep. 18, 2008

(54) Device and method for fixing an electrical lead

(76) Inventor: Gil Vardi, Town and Country, MO (US)

Correspondence Address: PATRICK W. RASCHE;ARMSTRONG TEASDALE LLP, ONE METROPOLITAN SQUARE, SUITE 2600, ST. LOUIS, MO, 63102-2740, US

(21) Appl. No.: 11/717,471

(22) Filed: Mar. 12, 2007

(51) Int. Cl. A61N 1/375

(52) U.S. Cl. 607/9

(57) ABSTRACT

An electrical lead for a cardiac pacing device includes a body including a tip portion defining a void. A fixation device is coupled to the body. The fixation device is at least partially positioned within the void in a compressed configuration and movable between the compressed configuration and a deployed configuration to facilitate fixing the electrical lead within the vessel.

### BACKGROUND OF THE INVENTION

This invention relates generally to the placement of an electrical lead for a cardiac pacemaker and, more particularly, to a device and method for fixing the electrical lead within a desired coronary vein.

Cardiac pacemakers are employed to treat cardiac conditions, such as arrhythmias, and provide assistance to a diseased or injured heart muscle. Conventional cardiac pacemakers include electrical leads positioned with respect to selected areas of the heart muscle to facilitate muscle stimulation and/or provide rhythmic muscle contractions.

A biventricular pacemaker, typically used in cardiac resynchronization therapy (CRT), paces the left ventricle as well as the right ventricle to synchronize a heart that does not beat in synchrony; a commonly occurring condition in patients that suffer from heart failure. The biventricular pacemaker includes an electrical lead positioned with respect to the atrium, an electrical lead positioned with respect to the right ventricle and at least one electrical lead positioned with respect to the left ventricle. More specifically, an electrical lead is routed through the coronary sinus and associated coronary veins and positioned with respect to the left ventricle to pace the left ventricle.

Due to the anatomical configuration and/or the dimensions of the coronary sinus and associated coronary veins on the left side of the heart, an electrical lead routed through this vasculature necessarily has suitably small dimensions relative to conventional electrical leads that are positioned with respect to the right ventricle. Further, these electrical leads are fixed within the vein and should be configured to withstand the adverse conditions within the vein as a result of blood flow and/or heart muscle motion or movement, for example.

### BRIEF DESCRIPTION OF THE INVENTION

In one aspect, an electrical lead for a cardiac pacing device is provided. The electrical lead includes a body including a tip portion defining a void. A fixation device is coupled to the body. The fixation device is at least partially positioned within the void in a compressed configuration and movable between the compressed configuration and a deployed configuration to facilitate fixing the electrical lead within the vessel.

In another aspect, an electrical lead for a cardiac pacing device is provided. The electrical lead includes a body and a fixation device coupled to the body. The fixation device is movable between a compressed configuration and a deployed configuration. A sheath is positioned about the fixation device to retain the fixation device in the compressed configuration. The sheath is movable with respect to the fixation device to facilitate deploying the fixation device. In the deployed configuration, the fixation device fixes the electrical lead within a vessel.

In another aspect, a method for fixing an electrical lead within a vessel is provided. The method includes introducing the electrical lead into the vessel with a fixation device of the electrical lead in a compressed configuration. The electrical lead is positioned at a selected position within the vessel and the fixation device is deployed to move to a deployed configuration. In the deployed configuration, the fixation device fixes the electrical lead within the vessel.

### BRIEF DESCRIPTION OF THE DRAWINGS

FIG. 1 is a side view of an electrical lead including an exemplary fixation device in a compressed configuration;

FIG. 2 is a side view of the electrical lead and the fixation device shown in FIG. 1 in a deployed configuration;

FIG. 3 is a side view of an electrical lead including an alternative exemplary fixation device in a compressed configuration;

FIG. 4 is a side view of the electrical lead and the fixation device shown in FIG. 3 in a deployed configuration;

FIG. 5 is a side view of an electrical lead including an alternative exemplary fixation device in a compressed configuration;

FIG. 6 is a side view of the electrical lead and the fixation device shown in FIG. 5 in a deployed configuration;

FIG. 7 is a side view of an electrical lead including an alternative exemplary fixation device in a compressed configuration;

FIG. 8 is a side view of the electrical lead and the fixation device shown in FIG. 7 in a deployed configuration;

FIG. 9 is a side view of an electrical lead including an alternative exemplary fixation device in a compressed configuration; and

FIG. 10 is a side view of the electrical lead and the fixation device shown in FIG. 9 in a deployed configuration.

### DETAILED DESCRIPTION OF THE INVENTION

The present invention provides an apparatus and method for fixing an electrical lead within a vessel. Although the present invention is described below in reference to its application in connection with an electrical lead for a cardiac pacing device that is routed through a patient's coronary sinus and fixedly positioned, temporarily or permanently, within an associated coronary vein, it should be apparent to those skilled in the art and guided by the teachings herein provided that the invention is likewise applicable for use with any suitable electrical lead or other device or component positioned within the patient's body.

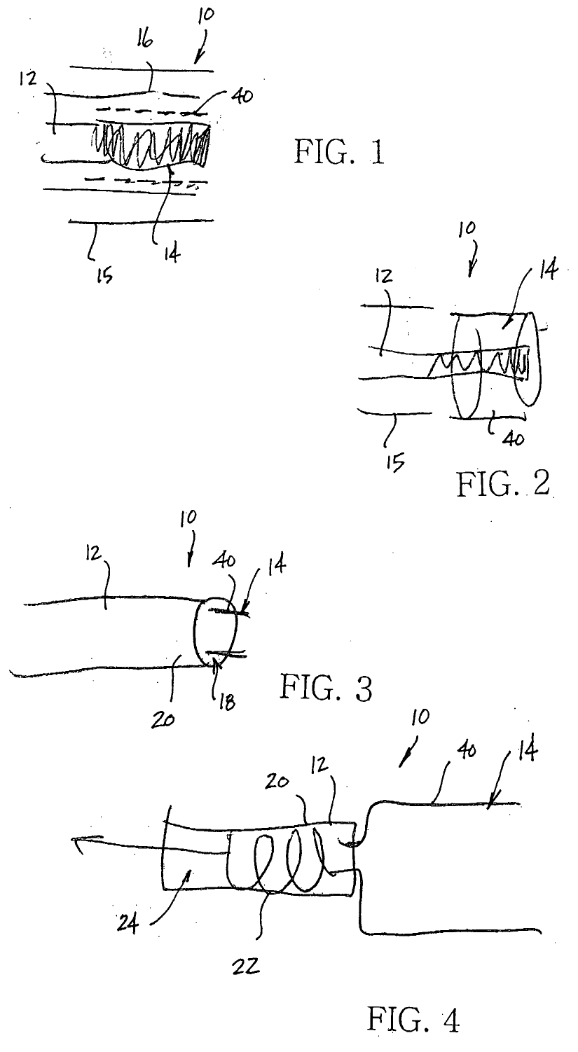

Referring to FIGS. 1-8, a medical device and/or system, such as a cardiac pacing device or pacemaker, includes a plurality of electrical leads 10. In one embodiment, the medical system is a cardiac pacing device that includes at least one electrical lead 10 that is fixedly positioned with respect to a patient's left ventricle to facilitate synchronizing a heart that does not beat in synchrony. The medical system includes, for example, an electrical lead positioned with respect to the atrium (not shown), an electrical lead positioned with respect to the right ventricle (not shown) and at least one electrical lead 10 positioned with respect to the left ventricle. More specifically, at least one electrical lead 10 is routed through the patient's coronary sinus and associated coronary vein(s) and fixedly positioned with respect to the left ventricle to pace the left ventricle. With electrical lead 10 properly positioned within the vessel, electrical lead 10 is fixed within the vessel, either permanently or temporarily. The electrical lead positioned with respect to the atrium and/or the electrical lead positioned with respect to the right ventricle may be the same or similar to or may be different from electrical lead 10.

In one embodiment, electrical lead 10 includes a body 12 having suitable dimensions to facilitate routing electrical lead 10 through the tortuous vasculature and a fixation device 14 coupled to body 12 to facilitate fixing electrical lead 10 within a vessel 15, such as a coronary vein. Fixation device 14 is movable between a compressed or retracted configuration and a deployed configuration. In the deployed configuration, fixation device 14 couples electrical lead 10 within the vessel. In one embodiment, a sheath 16, as described below, is positioned about fixation device 14 to retain fixation device 14 in the compressed configuration. Sheath 16 is movable with respect to fixation device 14 to facilitate deploying fixation device 14. In an alternative embodiment, at least a portion of fixation device 14 is positioned within a void 18 defined at a tip portion 20 of body 12 to retain fixation device 14 in the compressed configuration. A pusher 22 is configured to urge fixation device 14 from within void 18 to facilitate fixation device 14 moving from the compressed configuration to the deployed configuration. In a particular embodiment, at least a portion of pusher 22 is positioned within a passage 24 defined by body 12. Passage 24 is in communication with void 18. In one embodiment, pusher 22 is threadedly coupled to body 12 and rotatable within passage 24 to advance fixation device 14 towards tip portion 20. Alternatively, pusher 22 is slidably movable within passage 24.

In one embodiment, fixation device 14 is positioned within void 18 and/or coupled to body 12 prior to routing electrical lead 10 through the patient's vasculature. In an alternative embodiment, electrical lead 10 is routed through the patient's vasculature and properly positioned within vessel 15, such as a coronary vein. Fixation device 14 is then routed through passage 24 and void 18 in the retracted configuration. As fixation device 14 is urged through void 18, fixation device 14 moves toward the deployed configuration to couple electrical lead 10 within vessel 15.

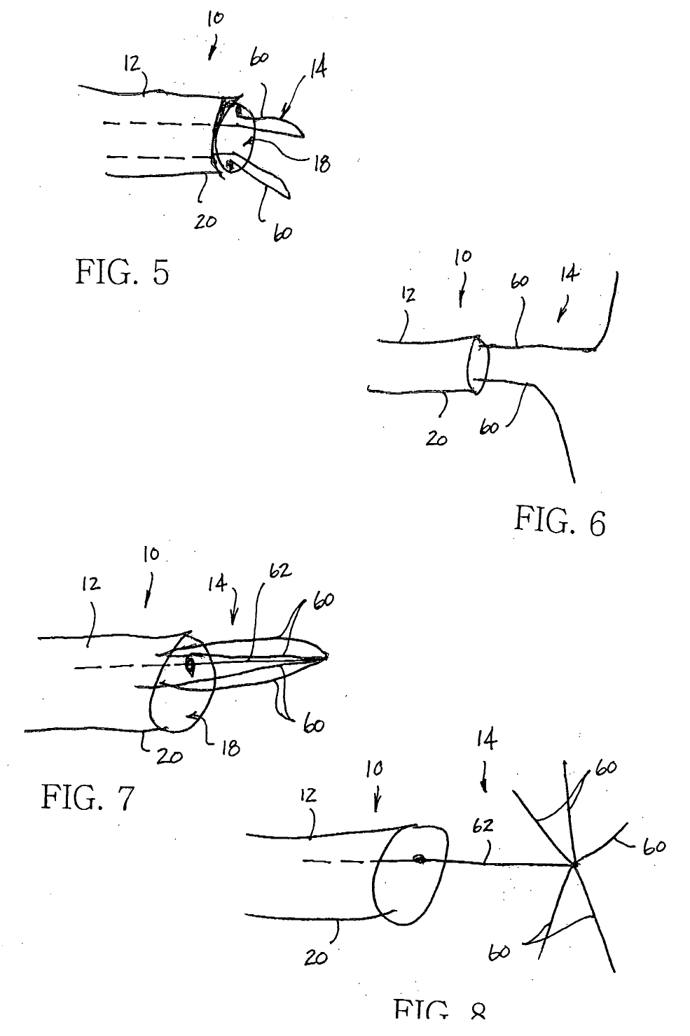

Referring further to FIGS. 1-4, in one embodiment, fixation device 14 includes a stent 40 coupled to body 12. Stent 40 is movable between a compressed configuration, as shown in FIGS. 1 and 3, and a deployed configuration, as shown in FIGS. 2 and 4. In a particular embodiment, stent 40 is biased towards the deployed configuration. In the deployed configuration, stent 40 fixes electrical lead 10 within vessel 15. In a further particular embodiment, stent 40 is coupled to tip portion 20 of body 12. As shown in FIG. 1, sheath 16 is positioned about at least a portion of stent 40 to retain stent 40 in the compressed configuration. Sheath 16 is movable with respect to stent 40 to facilitate deploying stent 40. Stent 40 expands radially outwardly with respect to body 12 towards the deployed configuration. In the deployed configuration, stent 40 contacts and/or interferes with an inner surface of vessel 15 to facilitate fixing electrical lead 10 within vessel 15.

In one embodiment, upon moving sheath 16 from about stent 40, stent 40 moves towards the deployed configuration. Stent 40 is made of a suitable biocompatible material including, without limitation, suitable metal materials, such as stainless steel, platinum, gold, titanium and nickel and/or composites or alloys thereof. In a particular embodiment, stent 40 is fabricated at least partially from a material having shape memory properties. Suitable materials include, without limitation, Nitinol and other known shape memory alloys (SMA) having properties that develop a shape memory effect (SME), which allows the material to return to an initial configuration after a force applied to the material to shape, stretch, compress and/or deform the material is removed. In a further embodiment, stent 40 is fabricated from a thermally treated metal alloy (TMA) including, without limitation, nickel titanium, beta titanium, copper nickel titanium and any combination thereof. It should be apparent to those skilled in the art and guided by the teachings herein provided that stent 40 may be made or fabricated using any suitable biocompatible material preferably, but not necessarily, having suitable shape memory properties. Further, stent 40 may have any suitable size, shape and/or configuration, which provide sufficient structural strength as required.

Referring further to FIGS. 3 and 4, in an alternative embodiment, stent 40 is positioned within void 18 defined at tip portion 20 of body 14. Stent 40 is at least partially positioned within void 18 in the compressed configuration such that stent 40 is retained in the compressed configuration to facilitate routing electrical lead 10 through the patient's vasculature. In one embodiment, a suitable mechanism, such as pusher 22, is configured to urge stent 40 from within void 18 to facilitate stent 40 moving towards the deployed configuration. As shown in FIG. 4, in one embodiment, pusher 22 is positioned within passage 24 at least partially defined through tip portion 20.

Referring further to FIGS. 5-8, in a further alternative embodiment fixation device 14 includes at least one strut 60 operatively coupled to body 12. Struts 60 are made of a suitable biocompatible material including, without limitation, materials as described above in reference to stent 40. In a particular embodiment, struts 60 are fabricated at least partially from a material having shape memory properties.

Each strut 60 is movable between a retracted configuration, as shown in FIGS. 5 and 7, and a deployed configuration, as shown in FIGS. 6 and 8. In one embodiment, strut 60 is bendable to facilitate moving between the retracted configuration and the deployed configuration. Alternatively or in addition, strut 60 is jointed to facilitate movement between the retracted configuration and the deployed configuration. In a particular embodiment, strut 60 is biased towards the deployed configuration. In the deployed configuration, each strut 60 extends radially outwardly from tip portion 20 and/or with respect to body 12 to couple to, contact and/or interfere with an inner surface of the vessel to fix electrical lead 10 within the vessel. As shown in FIGS. 5 and 7, strut 60 is initially positioned within void 18 defined by tip portion 20 of body 12. Strut 60 is at least partially positioned within void 18 in the compressed or retracted configuration to facilitate routing electrical lead 10 through the patient's vasculature. In one embodiment, a suitable mechanism or component, such as pusher 22, is configured to urge strut 60 from within void 18 to facilitate strut 60 moving to the deployed configuration. In the deployed configuration, struts 60 fix electrical lead 10 within the vessel. In a further embodiment, struts 60 are retractably movable from the deployed configuration to the retracted configuration to facilitate removing electrical lead 10 from within the vessel.

Referring further to FIGS. 7 and 8, in one embodiment, fixation device 14 has an umbrella configuration having a core 62 and a plurality of struts 60 that are movably coupled to core 62. In one embodiment, core 62 is substantially centrally located with respect to struts 60. In the retracted configuration as shown in FIG. 7, struts 60 are retained within void 18. As central core 62 is moved outwardly with respect to void 18, struts 60 open and move towards the deployed configuration, as shown in FIG. 8, to extend radially outwardly from central core 62.

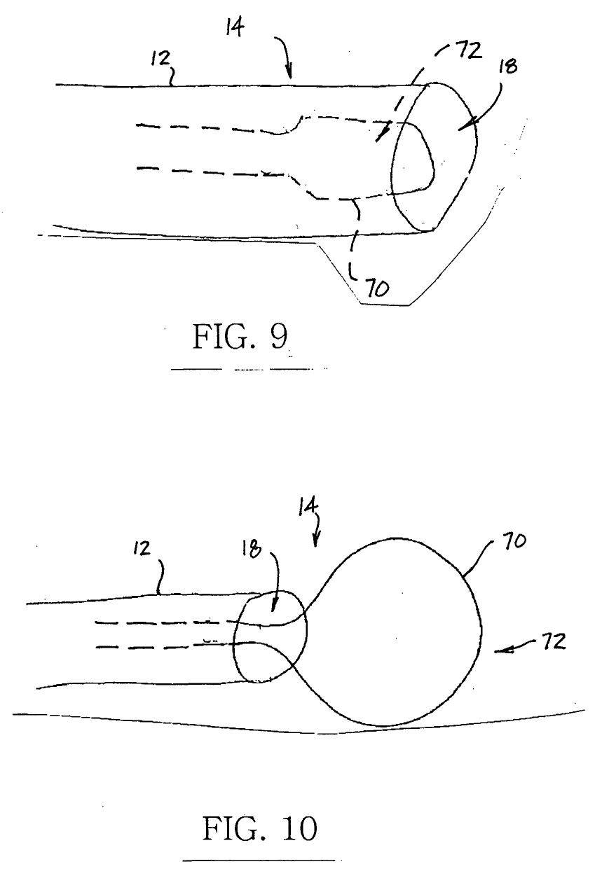

Referring further to FIGS. 9 and 10, in a further alternative embodiment fixation device 14 includes at least one wire 70 operatively coupled to body 12 and forming a loop portion 72. Wire 70 is made of a suitable biocompatible material including, without limitation, materials as described above in reference to stent 40 and/or struts 60. In a particular embodiment, wire 70 is fabricated at least partially from a material having shape memory properties.

Wire 70 is movable with respect to body 12 and within void 18 such that loop portion 72 is movable between a retracted or constricted configuration and at least partially positioned within void 18, as shown in FIG. 9, and a deployed or open configuration, as shown in FIG. 10. In one embodiment, at least a portion of wire 70, such as loop portion 72, is bendable or shapeable to facilitate moving loop portion 72 between the retracted configuration and the deployed configuration. Alternatively or in addition, at least a portion of wire 70 is jointed to facilitate movement of loop portion 72 between the retracted configuration and the deployed configuration. In a particular embodiment, loop portion 72 is biased towards the deployed configuration. Loop portion 72 moves towards the deployed configuration and opens or expands to couple to, contact and/or interfere with an inner surface of the vessel to fix electrical lead 10 within the vessel.

As shown in FIG. 9, loop portion 72 is initially positioned within void 18 defined by tip portion 20 of body 12. Loop portion 72 is at least partially positioned within void 18 in the compressed or retracted configuration to facilitate routing electrical lead 10 through the patient's vasculature. In one embodiment, a suitable mechanism or component, such as pusher 22, is configured to urge wire 70 from within void 18 to facilitate loop portion 72 moving towards the deployed configuration. In the deployed configuration, loop portion 72 fixes electrical lead 10 within the vessel. In a further embodiment, loop portion 72 is retractably movable from the deployed configuration to the retracted configuration to facilitate removing electrical lead 10 from within the vessel.

In one embodiment, a method for fixing electrical lead 10 within a vessel includes introducing electrical lead 10 into the vessel with fixation device 14 in a compressed or retracted configuration to facilitate routing the electrical lead 10 through the patient's vasculature. In a particular embodiment, electrical lead 10 is introduced into the vessel by routing electrical lead 10 through the coronary sinus and associated coronary vein(s). With electrical lead 10 at a selected position within the vessel, fixation device 14 moves towards the deployed configuration. In the deployed configuration, fixation device 14 fixes electrical lead 10 within the vessel.

In one embodiment, fixation device 14 includes stent 40 coupled to body 12. Stent 40 is actuated to move from the compressed configuration at least partially positioned within void 18 to the deployed configuration such that stent 40 expands radially outwardly to contact and/or interfere with the inner surface of the vessel and fix electrical lead 10 within the vessel. In this embodiment, sheath 16 is moved with respect to stent 40 such that stent 40 is able to move from the compressed configuration to the deployed configuration.

In an alternative embodiment, fixation device 14 includes at least one strut 60 that is operatively coupled to body 12. In the compressed or retracted configuration, each strut 60 is at least partially positioned within void 18 defined by tip portion 20 to retain struts 60 in the compressed or retracted configuration. Each strut 60 is actuated to move from the compressed or retracted configuration at least partially positioned within void 18 to a deployed configuration such that each strut 60 extends radially outwardly to couple to and/or interfere with the inner surface of the vessel to fix electrical lead 10 within the vessel. In a particular embodiment, each strut 60 is biased towards the deployed configuration.

In a further alternative embodiment, fixation device 14 includes at least one wire 70 that is operatively coupled to body 12. Wire 70 includes a loop portion 72 that, in the compressed or retracted configuration, is at least partially positioned within void 18 defined by tip portion 20 to retain loop portion 72 in the compressed configuration. Wire 70 is moved with respect to body 12 such that loop portion 72 is moved from the compressed configuration at least partially positioned within void 18 to a deployed configuration such that loop portion 72 opens to couple to and/or interfere with the inner surface of the vessel to fix electrical lead 10 within the vessel. In a particular embodiment, loop portion 72 is biased towards the deployed configuration.

The above-described apparatus and method for fixing an electrical lead within a vessel facilitates accurately positioning the electrical lead with respect to the patient's heart, such as with respect to the left ventricle, to facilitate pacing the heart.

Exemplary embodiments of an apparatus and method for fixing an electrical lead are described above in detail. The apparatus and method are not limited to the specific embodiments described herein, but rather, components of the apparatus and/or steps of the method may be utilized independently and separately from other components and/or steps described herein. Further, the described apparatus components and/or method steps can also be defined in, or used in combination with, other apparatus and/or methods, and are not limited to practice with only the apparatus and method as described herein.

While the invention has been described in terms of various specific embodiments, those skilled in the art will recognize that the invention can be practiced with modification within the spirit and scope of the claims.

What is claimed is:

1. An electrical lead for a cardiac pacing device, said electrical lead comprising a body including a tip portion defining a void, and a fixation device coupled to said body, said fixation device at least partially positioned within said void in a compressed configuration and movable between the compressed configuration and a deployed configuration to facilitate fixing said electrical lead within the vessel.

2. An electrical lead in accordance with claim 1 further comprising a pusher configured to urge said fixation device from within said void to facilitate said fixation device moving from the compressed configuration to the deployed configuration.

3. An electrical lead in accordance with claim 2 wherein at least a portion of said pusher is positioned within a passage defined by said body.

4. An electrical lead in accordance with claim 1 wherein said fixation device comprises a stent coupled to said tip portion, said stent expanded radially outwardly in the deployed configuration to contact an inner surface of the vessel.

5. An electrical lead in accordance with claim 1 wherein said fixation device comprises a stent at least partially positioned within said void in the compressed configuration.

6. An electrical lead in accordance with claim 5 further comprising a pusher configured to urge said stent from within said void to facilitate said stent moving to the deployed configuration.

7. An electrical lead in accordance with claim 1 wherein said fixation device comprises at least one strut operatively coupled to said body, said at least one strut at least partially positioned within said void in a retracted configuration and movable to the deployed configuration, in the deployed configuration said at least one strut extending radially outwardly with respect to said body to contact an inner surface of the vessel.

8. An electrical lead in accordance with claim 7 wherein said at least one strut is biased towards the deployed configuration.

9. An electrical lead in accordance with claim 7 further comprising a pusher configured to urge said at least one strut from within said void to facilitate said at least one strut moving to the deployed configuration.

10. An electrical lead in accordance with claim 1 wherein said fixation device comprises at least one wire operatively coupled to said body, said at least one wire comprising a loop portion at least partially positioned within said void in a retracted configuration and movable to the deployed configuration, in the deployed configuration said loop portion extending radially outwardly with respect to said body to contact an inner surface of the vessel.

11. An electrical lead in accordance with claim 10 wherein said loop portion is biased towards the deployed configuration.

12. An electrical lead in accordance with claim 10 further comprising a pusher configured to urge said at least one wire from within said void to facilitate said loop portion moving to the deployed configuration.

13. An electrical lead for a cardiac pacing device, said electrical lead comprising a body and a fixation device coupled to said body, said fixation device movable between a compressed configuration and a deployed configuration, and a sheath positioned about said fixation device to retain said fixation device in the compressed configuration, said sheath movable with respect to said fixation device to facilitate deploying said fixation device, in the deployed configuration said fixation device fixing said electrical lead within a vessel.

14. An electrical lead in accordance with claim 13 wherein said fixation device comprises a stent coupled to said body, said stent movable between the compressed configuration and the deployed configuration, in the deployed configuration said stent extending radially outwardly from said body to contact an inner surface of the vessel.

15. An electrical lead in accordance with claim 14 wherein said stent is coupled to a tip portion of said body.

16. A method for fixing an electrical lead within a vessel, said method comprising:
introducing the electrical lead into the vessel with a fixation device of the electrical lead in a compressed configuration;
positioning the electrical lead at a selected position within the vessel; and
deploying the fixation device to move to the deployed configuration, in the deployed configuration the fixation device fixing the electrical lead within the vessel.

17. A method in accordance with claim 16 wherein introducing the electrical lead into the vessel further comprises routing the electrical lead through a patient's coronary sinus.

18. A method in accordance with claim 16 wherein the fixation device further comprises a body and a stent coupled to the body, and deploying the fixation device further comprises actuating the stent to move from a compressed configuration to a deployed configuration, in the deployed configuration the stent fixing said electrical lead within the vessel.

19. A method in accordance with claim 18 further comprising moving a sheath positioned about the stent with respect to the stent to facilitate deploying the stent.

20. A method in accordance with claim 16 wherein the fixation device further comprises a body and at least one strut operatively coupled to the body, and deploying the fixation device further comprises moving the at least one strut from a retracted configuration at least partially positioned within the body to a deployed configuration, in the deployed configuration the at least one strut fixing the electrical lead within the vessel.

21. A method in accordance with claim 20 further comprising biasing the at least one strut towards the deployed configuration.

22. A method in accordance with claim 20 further comprising positioning at least a portion of the at least one strut within a void defined by a tip portion of the body to retain the at least one strut in the retracted configuration.

23. A method in accordance with claim 16 wherein the fixation device further comprises a body and at least one wire operatively coupled to the body, the wiring defining a loop portion, deploying the fixation device further comprises moving the loop portion from a retracted configuration at least partially positioned within the body to a deployed configuration, in the deployed configuration the loop portion fixing the electrical lead within the vessel.

24. A method in accordance with claim 23 further comprising biasing the loop portion towards the deployed configuration.

25. A method in accordance with claim 23 further comprising positioning at least a portion of the loop portion within a void defined by a tip portion of the body to retain the loop portion in the retracted configuration.
